(19) United States
(12) Patent Application Publication
(10) Pub. No.: US 2026/0029391 A1
(43) Pub. Date: Jan. 29, 2026

(54) SELF-CLEANING SMOKE DETECTOR

(71) Applicant: Microchip Technology Incorporated, Chandler, AZ (US)

(72) Inventors: Patrick McFarland, Gilbert, AZ (US); Arthur B. Eck, Gilbert, AZ (US)

(73) Assignee: Microchip Technology Incorporated, Chandler, AZ (US)

(21) Appl. No.: 18/924,322

(22) Filed: Oct. 23, 2024

Related U.S. Application Data
(60) Provisional application No. 63/674,498, filed on Jul. 23, 2024.

(51) Int. Cl. G01N 33/00; B08B 5/02; B08B 9/00

(52) U.S. Cl. CPC G01N 33/0029; B08B 5/02; B08B 9/00

(57) ABSTRACT

Various embodiments of the teachings herein include a monitoring system. An example includes: a housing defining an internal test chamber; one or more passageways allowing air flow into the internal test chamber from a surrounding area; a source of compressed gas; an air channel in the housing to direct gas from the source of compressed gas to dislodge contaminants in the one or more passageways; an actuator to activate the source of compressed gas; and a controller to operate the actuator.

### PRIORITY STATEMENT

This application claims priority to U.S. Provisional Patent Application No. 63/674,498 filed Jul. 23, 2024, the contents of which are hereby incorporated in their entirety.

### TECHNICAL FIELD

The present disclosure relates to monitoring systems. Various examples of the teachings herein include systems and/or methods for cleaning baffles in monitoring systems, e.g. smoke detectors.

### BACKGROUND

In the field of electronic devices, including monitors and sensors, a build-up of dust and debris may adversely affect the operation thereof. For example, such build-up may reduce the accuracy of sensor readings and the effectiveness of a monitor or a sensor element. The operation of environmental sensors such as smoke detectors and other life safety monitors may be compromised.

Some monitoring systems provide sensor elements mounted within a housing. That housing may include passageways allowing air flow into the housing so the sensor elements can come into contact with the air or elements carried by the air flow.

Some smoke detectors employ a light sensor which measures light reflected off smoke particles to identify the presence of smoke. This may include generating light with one component of the smoke detector and measuring it with another. Extraneous light impinging on a light sensor may interfere with accurate sensing. To avoid this, typical smoke detectors include a housing with baffles allowing smoke particles to enter a test chamber but reducing the entry of external light.

In a housing with passageways, baffles, or other vents restricting air flow, dust and debris may impede the travel of smoke particles into the test chamber or even block the passageways completely. Proper operation of a smoke detector may require periodic testing of its functions and ongoing maintenance, including replacing the housing and/or cleaning the passageways or baffles. Some manufacturers recommend periodic vacuuming of a smoke detector.

For the purposes of this disclosure, a monitor refers to an electronic device which monitors one or more conditions, such as a smoke detector or a thermostat. A sensor or sensor element refers to a specific element within such a monitor to detect a particular parameter or condition.

### BRIEF DESCRIPTION OF THE DRAWINGS

FIG. 1 illustrates an example monitoring system incorporating teachings of the present disclosure;

FIG. 2 illustrates the monitoring system of FIG. 1 in a partially exploded view;

FIG. 3 illustrates an example test chamber incorporating teachings of the present disclosure;

FIG. 4 illustrates a portion of an example monitoring system incorporating teachings of the present disclosure;

FIGS. 5A-5D illustrate various examples of flow patterns for a test chamber incorporating teachings of the present disclosure.

### DETAILED DESCRIPTION

The teachings of the present disclosure may be employed to reduce and/or eliminate the effect of dust and debris on a monitoring system. In some examples, the teachings include systems and/or methods to remove dust or debris from passageways leading into a housing or test chamber. The performance of a smoke detector may be affected by any resistance to air flow in the baffles or passageways caused by dust and/or debris. Some monitoring systems require human intervention to clean the baffles or otherwise remove dust and debris that may affect the performance of the monitor system. Some monitor systems may include an alert or alarm alerting a technician to clear the passageways and or replace the housing or baffle system. Some smoke detectors include vapor smoke canisters operated to perform self-testing without direct action by a technician.

Examples of the teachings herein include monitoring systems with channels providing fluid flow into a test chamber to direct air or another fluid to affect the removal of dust and debris, e.g., from baffles or other passageways providing access to an internal test chamber or to a sensor element mounted within the housing. Removing dust and debris from light baffles in a smoke detector may improve the flow of room air into the test chamber, including any smoke particles therein. The air flow directed by the channels may include air from a mechanical blower and/or a source of compressed air.

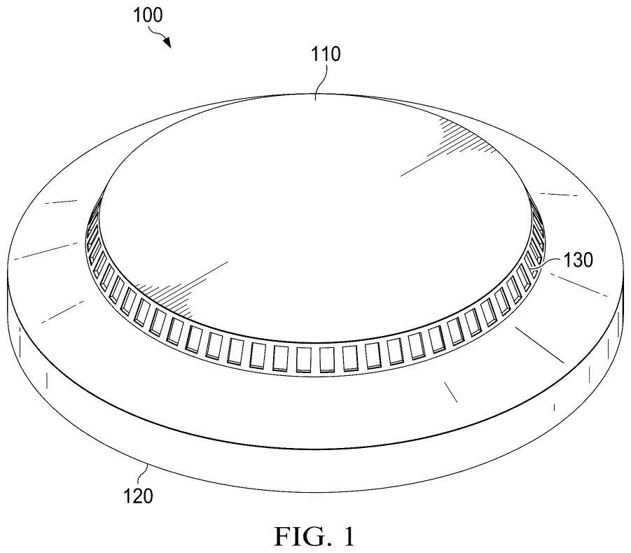

FIG. 1 illustrates an example monitoring system 100 incorporating teachings of the present disclosure. The monitoring system 100 includes an external housing top 110, an external housing bottom 120, and vents 130 allowing fluid flow into an interior defined between the external housing top 110 and the external housing bottom 120.

The monitoring system 100 may include one or more sensors, e.g., a smoke detector. In the example shown in FIG. 1, the monitoring system 100 includes an external housing bottom 120 to mount the system 100 to a wall or ceiling. In practice, the external housing bottom 120 may be mounted at the top of the system 100, e.g., mounted to the ceiling so the external housing top 110 actually hangs from the external housing bottom 120. In another example, the external housing bottom 120 may be mounted to a wall so the entire monitoring system 100 is rotated ninety degrees from the orientation shown in FIG. 1. The terms “top” and “bottom” are used relative to the orientation shown in FIG. 1 but do not limit the use of the components in practice.

As shown in FIG. 1, the external housing top 110 includes vents 130 to allow fluid flow into an interior space of the system 100. In practice, the vents 130 may be in any part of the housing, including the external housing bottom 120, or both. In practice, the vents 130 may be defined between two parts of the external housing.

The monitoring system 100 may include one or more sensor elements. The sensor elements may monitor any appropriate parameter and may operate under any appropriate scheme, including without limitation by measuring a capacitance, a current, a resistance, etc. The one or more sensor elements may be exposed to any air flow within a test chamber 110 and may, therefore, depend on air flow through the vents 130. In such a case, any blockage or impediment to air flow through the vents 130 may reduce the accuracy and/or efficiency of the monitoring system 100.

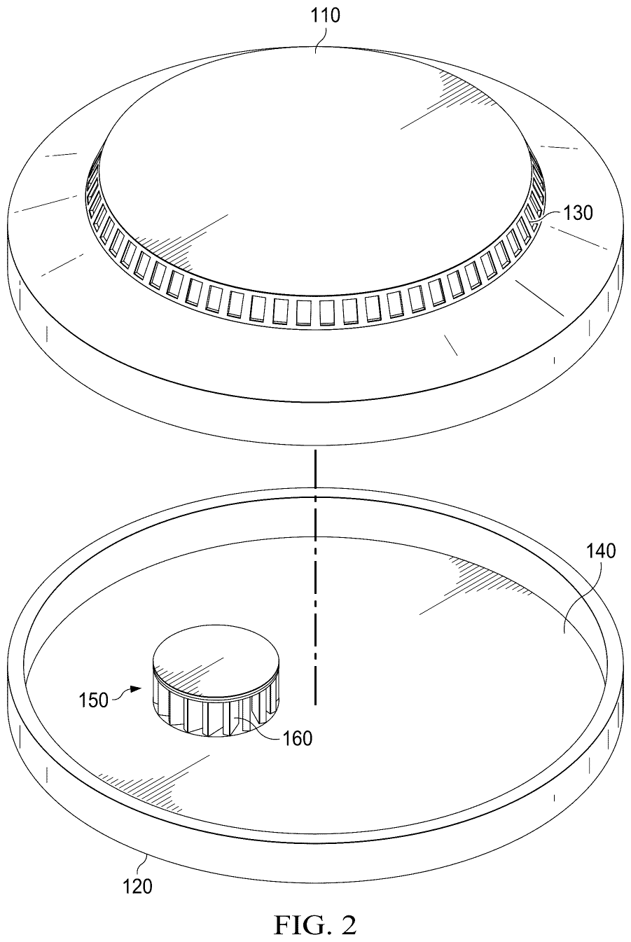

FIG. 2 illustrates an exploded view of the monitoring system 100. As shown in FIG. 2, the external housing top 110 and the external housing base 120 may be separate parts defining an interior. The monitoring system 100 includes a printed circuit board (PCB) 140. PCB 140 provides a mounting surface for an internal housing 150 defining a baffled test chamber. The internal housing 150 shown includes a set of passageways 160 allowing air or other fluid to flow from outside the internal housing 150 to an interior thereof.

In some systems, there may be a mounting surface that is not a PCB. For example, the internal housing 150 may be mounted directly to either the external housing top 110 or the external housing base 120. As another example, the internal housing 150 may be mounted to different elements of the system.

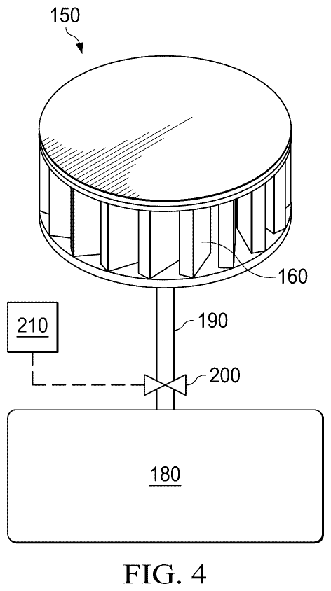

PCB 140 may include circuitry or leads to provide power and/or signals to components of the internal housing 150. As an example, a processor may be mounted to the PCB 140 and connected to the internal housing 150 by printed circuits or conductive tracks on the PCB 140 (described in more detail in relation to FIG. 4).

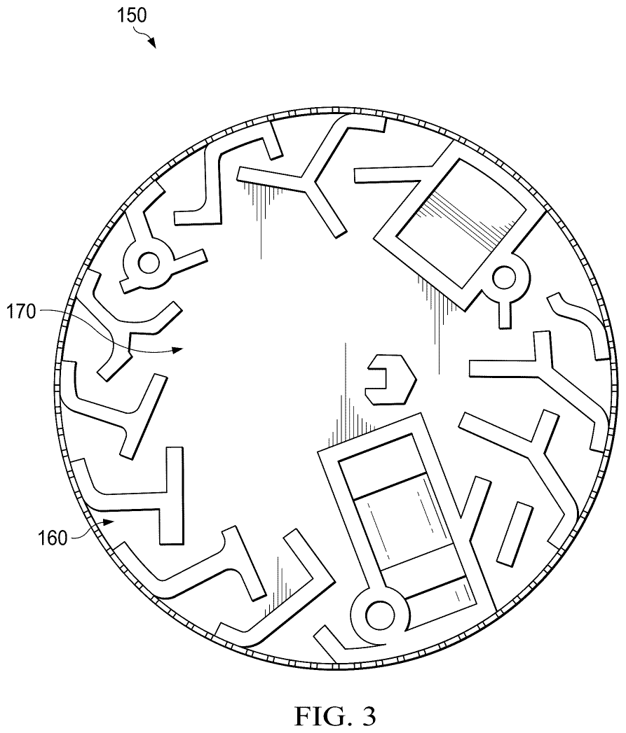

The internal housing 150 may include any combination of inlets or outlets appropriate for allowing air flow into the test chamber 110. As shown in FIG. 3, the internal housing 150 defines an internal test chamber for the monitoring system 100. The plurality of passageways 160 may include baffles configured to allow air flow (along with any entrained particles) into the test chamber 170 while restricting and/or blocking the entrance of light from outside the monitoring system 100. When the monitoring system 100 comprises a smoke detector, the baffles may deflect some or all ambient light from outside the monitoring system 100, providing a dark test chamber for a photochamber-style smoke detector. Some or all of the individual passageways 160 may become occluded with dust or other debris over time.

FIG. 4 is a schematic diagram showing an internal housing 150 with a plurality of passageways 160 similar to that shown in FIG. 3. In addition, the internal housing 150 in FIG. 4 has an associated source of compressed fluid 180. The compressed fluid stored in source 180 is in fluid communication with an interior of the internal housing 150 through an air channel 190. The flow of compressed fluid from the source 180 through the air channel 190 may be controlled, blocked, etc. by an actuator 200. Actuator 200 may be controlled and/or operated by a controller 210.

The monitoring system 100 may include one air channel 190 or a plurality of air channels 190, some or all of which may be at least in part integrated in the internal housing. The air channel 190 may be disposed to direct compressed fluid from the source 180 and thereby dislodge accumulated dust or debris from the passageways 160. The design and layout of the air channels 190 may be optimized for effective spray patterns and/or minimizing noise created during cleaning of the passageways 160.

The source 180 may include a canister holding compressed gas, e.g., carbon dioxide. In some examples, the source 180 may include elements to compress room air and use it as the compressed fluid as described herein.

The actuator 200 may include any element to activate the canister, including but not limited to a solenoid and/or a quick exhaust valve.

The actuator 200 may be controlled by a controller 210. The controller 210 may include a microcontroller otherwise in the monitoring system 100 or a smoke detection application specific integrated circuit (ASIC).

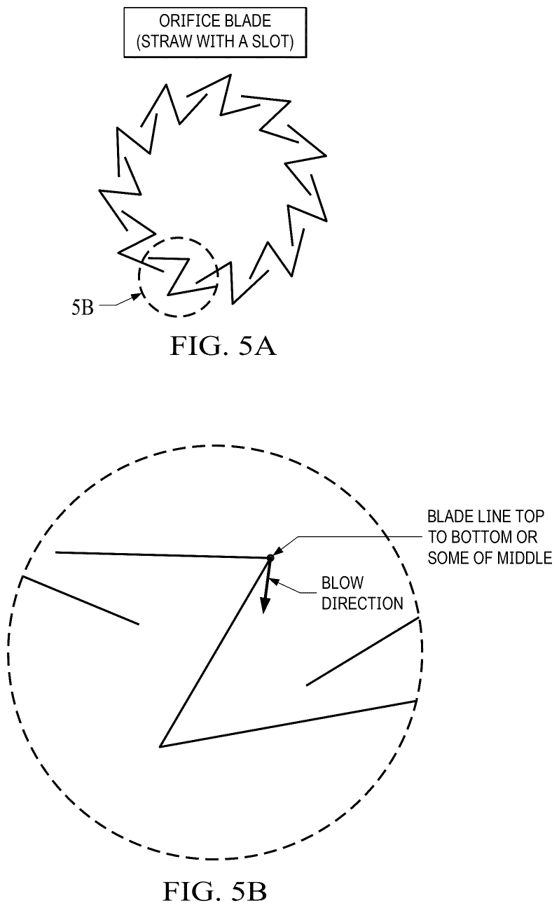

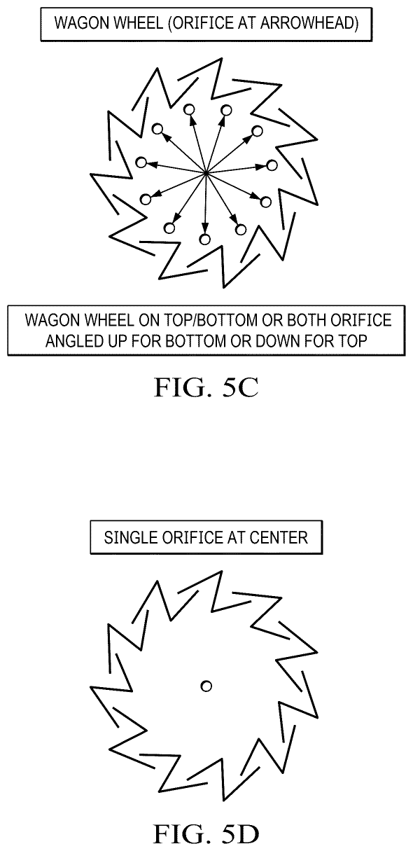

FIGS. 5A-5C illustrate various examples of flow patterns for a test chamber incorporating teachings of the present disclosure. These examples show a set of passageways comprising a generic baffle pattern. In each case, a flow of compressed fluid may enter the test chamber at one or more orifices and pass through one or more of the passageways. FIG. 5A, for example, shows an orifice blade which, as shown, may be present at a corner of one of the baffles, blowing the compressed fluid into one of the passageways. FIG. 5B shows a portion of the system from FIG. 5A highlighting the orifice blade.

FIG. 5C shows an array of orifices. The array of orifices may be fed by a so-called wagon wheel of air channels in the top and/or bottom of the test chamber. FIG. 5D shows a single orifice at the center of the test chamber.

The teachings of the present disclosure may extend the life span of the monitoring system 100. The teachings herein may reduce the frequency of technician interaction with the monitoring system 100 by, for example, increasing the period between replacement and/or manual maintenance. Further, the cleaning techniques taught herein may reduce the chances of a missed cleaning due to human error.

We claim:

1. A system comprising:
a housing defining an internal test chamber;
one or more passageways allowing air flow into the internal test chamber from a surrounding area;
a source of compressed gas;
an air channel in the housing to direct gas from the source of compressed gas to dislodge contaminants in the one or more passageways;
an actuator to activate the source of compressed gas; and
a controller to operate the actuator.

2. The system as recited in claim 1, wherein the source of compressed gas includes a canister.

3. The system as recited in claim 1, wherein the source of compressed gas includes a bellows.

4. The system as recited in claim 1, wherein the air channel leads from the source of compressed gas to the internal test chamber and the compressed gas increases a pressure in the internal test chamber.

5. The system as recited in claim 1, wherein the compressed gas includes air and/or carbon dioxide.

6. The system as recited in claim 1, wherein the air channel terminates in an orifice blade at a corner of a baffle in one of the passageways.

7. The system as recited in claim 1, wherein the air channel terminates at a wagon wheel including multiple orifices disposed in the housing.

8. The system as recited in claim 1, wherein the air channel terminates at a single orifice in a central portion of the housing.

9. A method for operating a monitor system, the method comprising:
triggering an actuator to release compressed gas from a source of compressed gas;
directing the gas from the source of compressed gas into an air channel; and
delivering the gas through the air channel to one or more passageways providing access to a test chamber in a housing.

10. The method as recited in claim 9, wherein the source of compressed gas includes a canister.

11. The method as recited in claim 9, wherein the source of compressed gas includes a bellows.

12. The method as recited in claim 9, wherein the air channel leads from the source of compressed gas to the internal test chamber and the compressed gas increases a pressure in the internal test chamber.

13. The method as recited in claim 9, wherein the compressed gas includes air and/or carbon dioxide.

14. The method as recited in claim 9, wherein the air channel terminates in an orifice blade at a corner of a baffle in one of the passageways.

15. The method as recited in claim 9, wherein the air channel terminates at a wagon wheel including multiple orifices disposed in the housing.

16. The method as recited in claim 9, wherein the air channel terminates at a single orifice in a central portion of the housing.
